(19) United States
(12) Patent Application Publication
(10) Pub. No.: US 2026/0234248 A1
(43) Pub. Date: Aug. 13, 2026

(54) Low dose antibody-based methods for treating hematologic malignancies

(71) Applicant: Actinium Pharmaceuticals, Inc., New York, NY (US)

(72) Inventor: Dragan Cicic, Brooklyn, NY (US)

(73) Assignee: Actinium Pharmaceuticals, Inc., New York, NY (US)

(21) Appl. No.: 19/300,849

(22) Filed: Aug. 15, 2025

Related U.S. Application Data
(63) Continuation of application No. 17/679,213, filed on Feb. 24, 2022, now Pat. No. 12,410,249.
Application No. 17/679,213 is a continuation of application No. 16/300,672, filed on May 25, 2017, now Pat. No. 11,292,835.
(60) Provisional application No. 62/342,568, filed on May 27, 2016.

(51) Int. Cl. C07K 16/28; A61K 39/00; A61K 51/10; A61P 35/02

(52) U.S. Cl. CPC C07K 16/2803; A61K 51/1069; A61K 51/1093; A61K 51/1096; A61P 35/02; A61K 2039/505

(57) ABSTRACT

This invention provides a method for treating a subject afflicted with a hematologic malignancy comprising administering to the subject an agent targeting a hematologic malignancy-associated antigen, wherein the subject has a low peripheral cancerous cell burden. This invention also provides a method for treating a subject afflicted with a hematologic malignancy and having a high peripheral cancerous cell burden, comprising (i) medically lowering the subject's peripheral cancerous cell burden, and (ii) while the subject's peripheral cancerous cell burden is still low, administering to the subject an agent targeting a hematologic malignancy-associated antigen. Particularly envisioned are the subject methods for treating acute myeloid leukemia using an anti-CD33 antibody labeled with an alpha-emitting isotope, such as ²²⁵Ac-HuM195.

This application is a continuation of U.S. Ser. No. 17/679,213, filed Feb. 24, 2022, which is a continuation of U.S. Ser. No. 16/300,672, filed Nov. 12, 2018, which is a § 371 national stage entry of PCT Application No. PCT/US2017/034406, filed May 25, 2017, which claims priority of U.S. Provisional Application No. 62/342,568, filed May 27, 2016, the contents of all of which are incorporated herein by reference in their entirety.

Throughout this application, various publications are cited. The disclosure of these publications is hereby incorporated by reference into this application to describe more fully the state of the art to which this invention pertains.

### SEQUENCE LISTING

The instant application contains a Sequence Listing which has been submitted electronically in XML format and is hereby incorporated by reference in its entirety. Said XML copy, created on Apr. 30, 2026, is named Actinium-1PCON2_SL.xml and is 13,111 bytes in size.

### FIELD OF THE INVENTION

The present invention relates to treating a subject afflicted with a hematologic malignancy using a low dose of a potent agent (such as a toxin-conjugated antibody) targeting a hematologic malignancy-associated antigen, wherein the subject has a low peripheral cancerous cell burden.

### BACKGROUND OF THE INVENTION

Cytotoxic agent-conjugated antibodies have recently become a promising tool for treating hematologic malignancies such as acute myeloid leukemia (“AML”). Of particular interest are therapeutic conjugates of cancer cell-specific antibodies with potent cytotoxic agents like alpha-emitting isotopes such as 225-actinium (²²⁵Ac).

However, doses of monoclonal antibodies labeled with potent cytotoxic agents cannot safely be escalated above maximum tolerated doses established in clinical trials. This safety-based dosing constraint prevents the escalation of antibody conjugate doses to saturation levels.

Therefore, for these conjugates, there is an unmet need for treating AML and other hematologic cancers by administering them in doses low enough to avoid toxicity while high enough to be therapeutically effective. There is also an unmet need to achieve this balance in a manner independent of patient age, co-morbidities and disease severity.

### SUMMARY OF THE INVENTION

This invention provides a method for treating a subject afflicted with a hematologic malignancy comprising administering to the subject an agent targeting a hematologic malignancy-associated antigen, wherein the subject has a low peripheral cancerous cell burden.

This invention also provides a method for treating a subject afflicted with a hematologic malignancy and having a high peripheral cancerous cell burden, comprising (i) medically lowering the subject's peripheral cancerous cell burden, and (ii) while the subject's peripheral cancerous cell burden is still low, administering to the subject an agent targeting a hematologic malignancy-associated antigen.

This invention further provides a method for treating a human subject afflicted with acute myeloid leukemia comprising administering to the subject an anti-CD33 antibody labeled with an alpha-emitting isotope, wherein (i) the subject has a low peripheral blast burden, and (ii) the antibody is administered in sub-saturating dose.

This invention still further provides a method for treating a human subject afflicted with acute myeloid leukemia and having a high peripheral blast burden, comprising (i) medically lowering the subject's peripheral blast burden, and (ii) while the subject's peripheral blast burden is still low, administering to the subject an anti-CD33 antibody labeled with an alpha-emitting isotope, wherein the antibody is administered in a sub-saturating dose.

This invention still further provides a method for treating a human subject afflicted with acute myeloid leukemia comprising intravenously administering ²²⁵Ac-labeled HuM195 to the subject, wherein (i) the subject has a low peripheral blast burden, and (ii) the ²²⁵Ac-labeled HuM195 is administered in a sub-saturating dose.

Finally, this invention provides a method for treating a human subject afflicted with acute myeloid leukemia and having a high peripheral blast burden, comprising (i) medically lowering the subject's peripheral blast burden, and (ii) while the subject's peripheral blast burden is still low, intravenously administering ²²⁵Ac-labeled HuM195 to the subject, wherein the ²²⁵Ac-labeled HuM195 is administered in a sub-saturating dose.

### BRIEF DESCRIPTION OF THE FIGURES

FIG. 1

This figure shows a schematic diagram of the expression plasmids for HuM195. The humanized VL and VH exons of HuM195 are flanked by Xbal sites. The VL exon was inserted into mammalian expression vector pVk, and the VH exon into pVg1 (Co, et al., J. Immunol. 148:1149-1154, 1992).

FIG. 2

This figure shows the complete sequence of the HuM195 light chain gene cloned in pVk between the Xbal and BamHI sites. The nucleotide number indicates its position in the plasmid pVk-HuM195. The VL and CK exons are translated in single letter code; the dot indicates the translation termination codon. The mature light chain begins at the double-underlined aspartic acid (D). The intron sequence is in italics. The polyA signal is underlined. Figure discloses SEQ ID NOS 1-2, respectively, in order of appearance.

FIG. 3

This figure shows the complete sequence of the HuM195 heavy chain gene cloned in pVg1 between the Xbal and BamHI sites. The nucleotide number indicates its position in the plasmid pVg1-HuM195. The VH, CH1, H, CH2 and CH3 exons are translated in single letter code; the dot indicates the translation termination codon. The mature heavy chain begins at the double-underlined glutamine (Q). The intron sequences are in italics. The polyA signal is underlined. Figure discloses SEQ ID NOS 3-4, respectively, in order of appearance.

FIG. 4

This figure shows the structure of ²²⁵Ac-Lintuzumab (²²⁵Ac-HuM195).

FIG. 5

This figure shows a flowchart for the production of ²²⁵Ac-HuM195.

FIG. 6

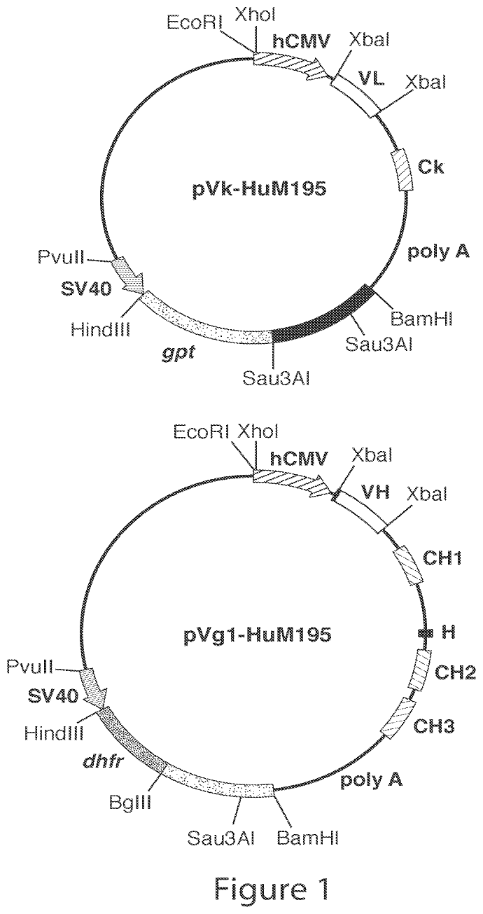

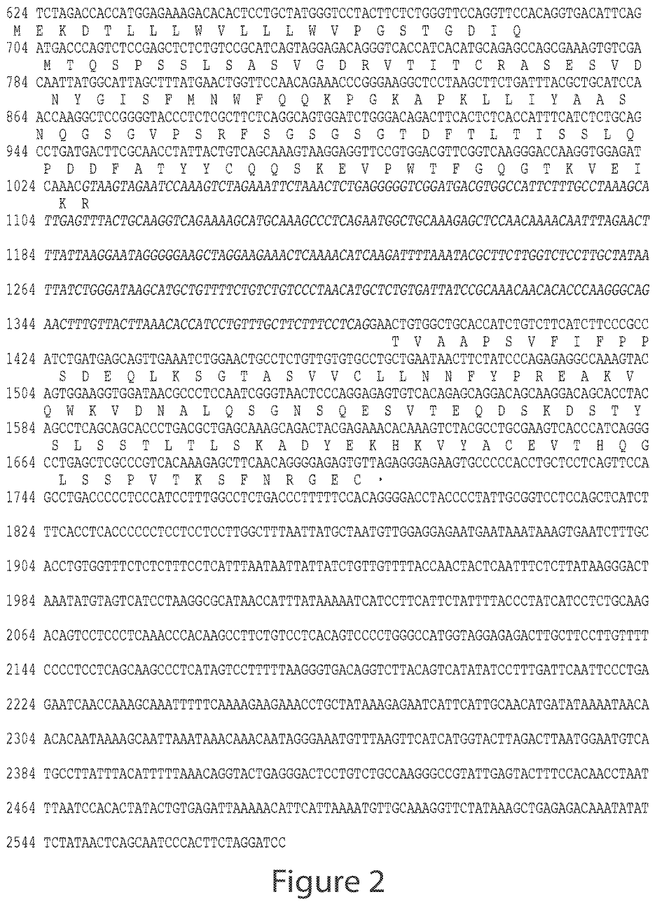

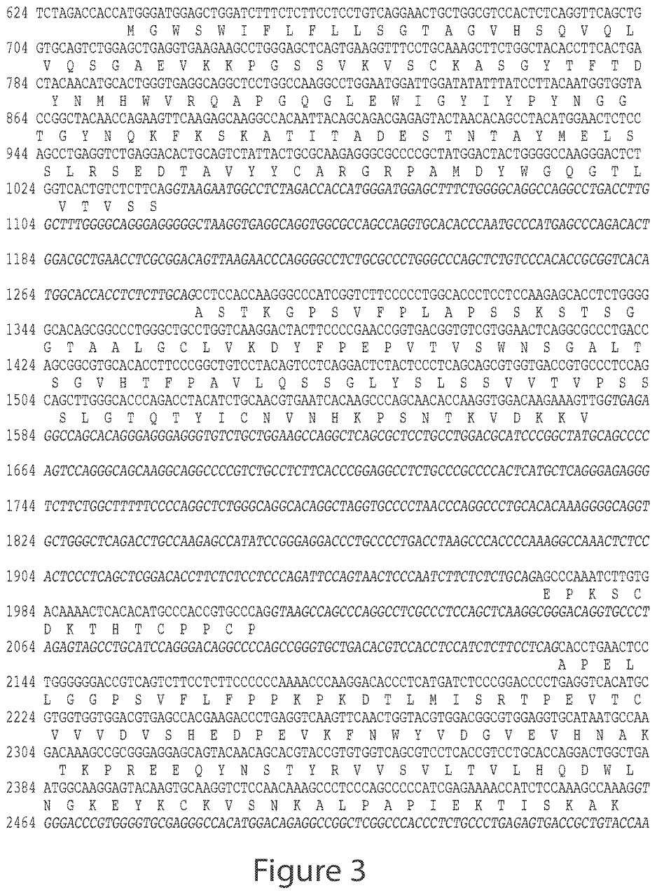

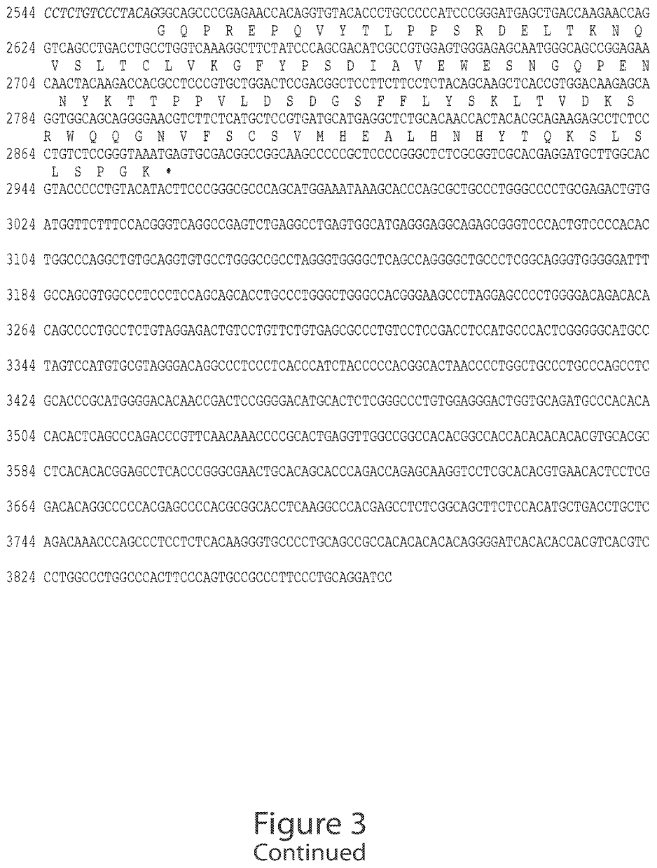

This figure shows a dosing protocol for ²²⁵Ac-Lintuzumab (²²⁵Ac-HUM195) treatment of AML.

### DETAILED DESCRIPTION OF THE INVENTION

This invention provides surprisingly effective methods for treating a subject afflicted with a hematologic malignancy, such as AML. These methods use a potent agent, such as an antibody conjugated with an alpha-emitting isotope, targeting a hematologic malignancy-associated antigen, such as CD33. The subject who is being treated has a low peripheral cancerous cell burden and, ideally, only a sub-saturating dose of agent is needed to treat the subject.

### Definitions

In this application, certain terms are used which shall have the meanings set forth as follows.

As used herein, “administer”, with respect to an agent, means to deliver the agent to a subject's body via any known method. Specific modes of administration include, without limitation, intravenous, oral, sublingual, transdermal, subcutaneous, intraperitoneal, intrathecal and intra-tumoral administration.

In addition, in this invention, the various antibodies and other antigen-targeting agents used can be formulated using one or more routinely used pharmaceutically acceptable carriers. Such carriers are well known to those skilled in the art. For example, injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

As used herein, the “agent” targeting a hematologic malignancy-associated antigen can be any type of compound or composition useful for such purpose. Types of agents include, without limitation, antibodies, other protein-based drugs, peptides, nucleic acids, carbohydrates and small molecules drugs.

As used herein, the term “antibody” includes, without limitation, (a) an immunoglobulin molecule comprising two heavy chains and two light chains and which recognizes an antigen; (b) polyclonal and monoclonal immunoglobulin molecules; (c) monovalent and divalent fragments thereof, and (d) bi-specific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. Antibodies can be both naturally occurring and non-naturally occurring. Furthermore, antibodies include chimeric antibodies, wholly synthetic antibodies, single chain antibodies, and fragments thereof. Antibodies may be human, humanized or nonhuman.

As used herein, an “anti-CD33 antibody” is an antibody that binds to any available epitope of CD33. In one embodiment, the anti-CD33 antibody binds to the epitope recognized by the antibody HuM195.

As used herein, the term “burden”, when used in connection with a cancerous cell, means quantity. So, a cancerous cell “burden” means the quantity of cancerous cells. Cancerous cells have a burden with respect to their tissue of origin (i.e., the primary site of disease), such as the “bone marrow blast burden” in the case of AML. Cancerous cells also have a burden with respect to one or more tissues other than those of origin, such as the blast burden in blood, liver and spleen in the case of AML. The term “peripheral burden” relates to such cells. The peripheral burden of cancerous cells, such as blasts in the case of AML, can be measured in different ways with different outcomes. For example, in the case of AML, the “peripheral blast burden” can be measured as the total blast population outside of the bone marrow, or the total blast population of the blood, spleen and liver combined, or simply the blast population of the blood as measured in cells per unit volume. As used herein in connection with AML and other cancers originating in the bone marrow, and unless stated otherwise, the term “peripheral cancerous cell burden” (e.g., peripheral blast burden) refers to the cancerous cell population of the blood as measured in cells per unit volume (e.g., cells/μl). This blood-based measurement is a useful proxy for the more cumbersome measurements of spleen and liver burdens, for example.

Herein, a peripheral cancerous cell burden in a subject is “high” if, when the subject is administered an agent (e.g., an antibody) targeting a hematologic malignancy-associated antigen at the maximum safe dose, the agent does not reach the primary site of disease in a sufficient amount to bind to more than 90% of its target antigens at that site. Conversely, a peripheral cancerous cell burden in a subject is “low” if, when the subject is administered an agent (e.g., an antibody) targeting a hematologic malignancy-associated antigen at the maximum safe dose, the agent reaches the primary site of disease in a sufficient amount to bind to more than 90% of its target antigens at that site. In the case of AML, examples of low peripheral blast burden are those yielding blood blast burdens at or below 1,000 blast cells/μl, at or below 500 blast cells/μl, at or below 400 blast cells/μl, at or below 300 blast cells/μl, at or below 200 blast cells/μl, at or below 100 blast cells/μl, and at or below 50 blast cells/μl.

As used herein, the term “cytotoxic agent” includes, without limitation, a radionuclide, a protein-based toxin, a non-protein based toxin, and a chemotherapeutic agent. Radionuclides include, for example, alpha-emitting isotopes (i.e., alpha-emitting isotopes, such as ²²⁵Ac, ²¹³Bi and ²¹³Po), beta-emitting isotopes (e.g., ⁹⁰Y), and gamma-emitting isotopes (e.g., ¹³⁷Cs (which emits gamma rays via its decay product ¹³⁷Ba)). Protein-based toxins include, without limitation, ricin and toxic portions thereof, and botulinum toxin and toxic portions thereof. Methods for affixing a cytotoxic agent to an antibody (i.e., “labeling” an antibody with a cytotoxic agent) are well known.

A “hematologic malignancy”, also known as a blood cancer, is a cancer that originates in blood-forming tissue, such as the bone marrow or other cells of the immune system. Hematologic malignancies include, without limitation, leukemias (such as AML, acute promyelocytic leukemia, acute lymphoblastic leukemia, acute mixed lineage leukemia, chronic myeloid leukemia, chronic lymphocytic leukemia, hairy cell leukemia, large granular lymphocytic leukemia), myelodysplastic syndrome (MDS), myeloproliferative disorders (polycitermia vera, essential thrombocytosis, primary myelofibrosis and chronic myeloid leukemia), lymphomas, multiple myeloma, and MGUS and similar disorders.

As used herein, a “hematologic malignancy-associated antigen” can be, for example, a protein and/or carbohydrate marker found exclusively or predominantly on the surface of a cancer cell associated with that particular malignancy. Examples of hematologic malignancy-associated antigens include, without limitation, CD20, CD33, CD38, CD45, CD52, CD123 and CD319.

The antibody “HuM195” (also known as lintuzumab) is known, as are methods of making it. Likewise, methods of labeling HuM195 with ²²⁵Ac are known. These methods are exemplified, for example, in Scheinberg, et al., U.S. Pat. No. 6,683,162. This information is also exemplified in the examples and figures below.

A low peripheral cancerous cell burden can be “medically induced” using any known method for doing so. These methods include, for example, pharmaceutical induction (such as by oral administration of hydroxyurea at 10-70 mg/kg/day), and spleen removal.

As used herein, the term “subject” includes, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 60 years or older, 65 or older, 70 or older, 75 or older, 80 or older, 85 or older, or 90 or older. For a human subject afflicted with AML, the subject's bone marrow blast burden can be, for example, over 5%, over 10%, over 20%, over 30%, over 40%, over 50%, over 60%, over 70%, over 80%, or over 90%. As an additional example, for a human subject afflicted with AML, the subject can be newly diagnosed, or relapsed and/or refractory, or in remission.

As used herein, a “sub-saturating dose” of an agent targeting a hematologic malignancy-associated antigen is one that introduces into the subject's body fewer target antigen-binding sites (e.g., Fab's) than there are target antigens. By way of example, for an anti-CD33 antibody, a sub-saturating dose is one that introduces into the subject's body fewer CD33-binding sites than there are CD33 molecules. In one embodiment, a sub-saturating dose of an agent targeting a hematologic malignancy-associated antigen is one where the ratio of target antigen-binding sites to target antigens is less than or equal to 9:10. In another embodiment, the ratio of target antigen-binding sites to target antigens is less than or equal to 1:2, less than or equal to 1:5, less than or equal to 1:10, less than or equal to 1:20, or less than or equal to 1:100.

For an agent such as an antibody labeled with an alpha-emitting isotope or other cytotoxic agent, the majority of the drug administered to a subject typically consists of non-labeled antibody, with the minority being the labeled antibody. Thus, in one embodiment, a sub-saturating dose of an agent targeting a hematologic malignancy-associated antigen is one where the ratio of total (i.e., labeled and unlabeled) target antigen-binding sites to target antigens is less than or equal to 9:10 (and can be less than or equal to 1:2, less than or equal to 1:5, less than or equal to 1:10, less than or equal to 1:20, or less than or equal to 1:100). In another embodiment, a sub-saturating dose of an agent targeting a hematologic malignancy-associated antigen is one where the ratio of labeled target antigen-binding sites to target antigens is less than or equal to 9:10 (and can be less than or equal to 1:2, less than or equal to 1:5, less than or equal to 1:10, less than or equal to 1:20, or less than or equal to 1:100).

Sub-saturating doses used in connection with this invention include, for example, a single administration, and two or more administrations (i.e., fractions). The amount administered in each dose can be measured, for example, by radiation (e.g., μCi/kg) or weight (e.g., mg/kg or mg/m²). In the case of ²²⁵Ac-HUM195 (also known as “Actimab-A”) for treating AML, dosing regimens include the following, without limitation: (i) 2×0.5 μCi/kg, 2×1.0 μCi/kg, 2×1.5 μCi/kg, or 2×2.0 μCi/kg, where the fractions are administered one week apart; (ii) 1×0.5 Ci/kg, 1×1.0 μCi/kg, 1×2.0 μCi/kg, 1×3.0 μCi/kg, or 1×4.0 μCi/kg; (iii) 1×15-20 μg/kg (0.03-0.06 μg/kg labeled); and (iv) less than or equal to approximately 2 mg per subject (approximately 0.04 mg labeled antibody per subject).

As used herein, “treating” a subject afflicted with a disorder shall include, without limitation, (i) slowing, stopping or reversing the disorder's progression, (ii) slowing, stopping or reversing the progression of the disorder's symptoms, (iii) reducing the likelihood of the disorder's recurrence, and/or (iv) reducing the likelihood that the disorder's symptoms will recur. In the preferred embodiment, treating a subject afflicted with a disorder means (i) reversing the disorder's progression, ideally to the point of eliminating the disorder, and/or (ii) reversing the progression of the disorder's symptoms, ideally to the point of eliminating the symptoms and/or (iii) reducing or eliminating the likelihood of relapse (i.e. consolidation, which is a common goal of post remission therapy for AML and, ideally, results in the destruction of any remaining leukemia cells).

The treatment of hematologic malignancy, such as the treatment of AML, can be measured according to a number of clinical endpoints. These include, without limitation, survival time (such as weeks, months or years of improved survival time, e.g., one, two or more months of additional survival time), and response status (such as complete remission (CR), near complete remission (nCR), very good partial remission (VGPR) and partial remission (PR)).

In one embodiment, treatment of hematologic malignancy, such as the treatment of AML, can be measured in terms of remission. Included here are the following non-limiting examples. (1) Morphologic complete remission (“CR”): ANC ≥1,000/mcl, platelet count ≥100,000/mcl, <5% bone marrow blasts, no Auer rods, no evidence of extramedullary disease. (No requirements for marrow cellularity, hemoglobin concentration). (2) Morphologic complete remission with incomplete blood count recovery (“CRi”): Same as CR but ANC may be <1,000/mcl and/or platelet count <100,000/mcl. (3) Partial remission (PR): ANC ≥1,000/mcl, platelet count >100,000/mcl, and at least a 50% decrease in the percentage of marrow aspirate blasts to 5-25%, or marrow blasts <5% with persistent Auer rods. These criteria and others are known, and are described, for example, in SWOG Oncology Research Professional (ORP) Manual Volume I, Chapter 11A, Leukemia (2014).

### Embodiments of the Invention

This invention provides a first method for treating a subject afflicted with a hematologic malignancy comprising administering to the subject an agent targeting a hematologic malignancy-associated antigen, wherein the subject has a low peripheral cancerous cell burden.

In one embodiment of the first method, the subject's low peripheral cancerous cell burden is medically induced, such as by administering a pharmaceutical agent like hydroxyurea. Alternatively, the subject's low peripheral cancerous cell burden can be naturally occurring.

This invention also provides a second method for treating a subject afflicted with a hematologic malignancy and having a high peripheral cancerous cell burden, comprising (i) medically lowering the subject's peripheral cancerous cell burden, and (ii) while the subject's peripheral cancerous cell burden is still low, administering to the subject an agent targeting a hematologic malignancy-associated antigen.

Preferably, in these methods, the subject is human. In one embodiment of these methods, the hematologic malignancy is acute myeloid leukemia, and the cancerous cells are leukemic blasts.

The antigen-targeting agent is preferably administered in a sub-saturating dose, and can be administered intravenously or via another route. This agent can be, for example, an anti-CD33 antibody labeled with a cytotoxic agent (e.g., an alpha-emitting isotope like ²²⁵Ac).

This invention further provides a third method for treating a human subject afflicted with acute myeloid leukemia comprising administering to the subject an anti-CD33 antibody labeled with an alpha-emitting isotope, wherein (i) the subject has a low peripheral blast burden, and (ii) the antibody is administered in a sub-saturating dose.

In one embodiment, the subject's low peripheral blast burden is medically induced, and is preferably pharmaceutically induced (e.g., via administering hydroxyurea). Alternatively, the subject's low peripheral blast burden can be naturally occurring.

This invention still further provides a fourth method for treating a human subject afflicted with acute myeloid leukemia and having a high peripheral blast burden, comprising (i) medically lowering the subject's peripheral blast burden, and (ii) while the subject's peripheral blast burden is still low, administering to the subject an anti-CD33 antibody labeled with an alpha-emitting isotope, wherein the antibody is administered in a sub-saturating dose.

Preferably, medically lowering the subject's peripheral blast burden comprises pharmaceutically lowering the subject's peripheral blast burden (e.g., via administering hydroxyurea). The antibody can be administered intravenously or via another route.

This invention still further provides a method for treating a human subject afflicted with acute myeloid leukemia comprising intravenously administering ²²⁵Ac-labeled HuM195 to the subject, wherein (i) the subject has a low peripheral blast burden, and (ii) the ²²⁵Ac-labeled HuM195 is administered in a sub-saturating dose.

In one embodiment, the subject's low peripheral blast burden is medically induced, and is preferably pharmaceutically induced (e.g., via administering hydroxyurea). Alternatively, the subject's low peripheral cancerous cell burden can be naturally occurring. The ²²⁵Ac-labeled HuM195 is preferably administered intravenously.

Finally, this invention provides a method for treating a human subject afflicted with acute myeloid leukemia and having a high peripheral blast burden, comprising (i) medically lowering the subject's peripheral blast burden, and (ii) while the subject's peripheral blast burden is still low, intravenously administering ²²⁵Ac-labeled HuM195 to the subject, wherein the ²²⁵Ac-labeled HuM195 is administered in a sub-saturating dose.

Preferably, medically lowering the subject's peripheral blast burden comprises pharmaceutically lowering the subject's peripheral blast burden (e.g., via administering hydroxyurea). Also, the ²²⁵Ac-labeled HuM195 is preferably administered intravenously.

This invention will be better understood by reference to the examples which follow, but those skilled in the art will readily appreciate that the specific examples detailed are only illustrative of the invention as described more fully in the claims which follow thereafter.

### Examples

### Example 1—Structure of ²²⁵Ac-Lintuzumab (²²⁵Ac-HuM195)

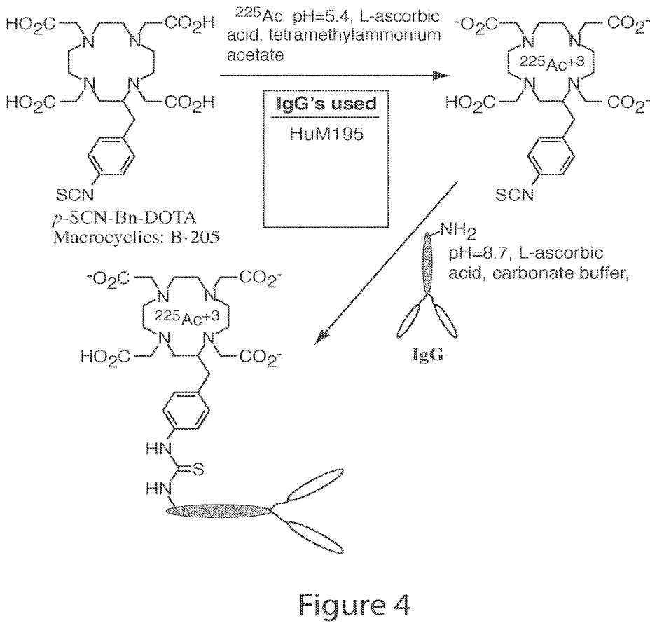

²²⁵Ac-Lintuzumab includes three key components; humanized monoclonal antibody HuM195 (generic name, lintuzumab), the alpha-emitting radioisotope ²²⁵Ac, and the bi-functional chelate 2-(p-isothiocyanatobenzyl)-1,4,7,10-tetraazacyclododecane-1,4,7,10-tetraacetic acid (p-SCN-Bn-DOTA). As depicted in FIG. 4, HuM195 is radiolabeled using the bi-functional chelate p-SCN-Bn-DOTA that binds to ²²⁵Ac and that is covalently attached to the IgG via a lysine residue on the antibody.

### Example 2—p-SCN-Bn-DOTA

DOTA, 2-(4-Isothiocyanatobenzyl)-1,4,7,10-tetraazacyclododecane tetraacetic acid (Macrocyclics item code B205-GMP) is synthesized by a multi-step organic synthesis that is fully described in U.S. Pat. No. 4,923,985.

### Example 3—Preparation of ²²⁵Ac-Lintuzumab (²²⁵Ac-HuM195)

The procedure for preparing ²²⁵Ac-Lintuzumab is based on the method described by Michael R. McDevitt, “Design and synthesis of ²²⁵Ac radioimmunopharmaceuticals”, Applied Radiation and Isotope, 57 (2002), 841-847. The procedure involves radiolabeling the bi-functional chelate, p-SCN-Bn-DOTA, with the radioisotope ²²⁵Ac, followed by binding of the radiolabeled p-SCN-Bn-DOTA to the antibody (HuM195). The construct, ²²⁵Ac-p-SCN-Bn-DOTA-HuM195, is purified using 10 DG size exclusion chromatography and eluted with 1% human serum albumin (HSA). The resulting drug product, Ac225-Lintuzumab, is then passed through a 0.2 μm sterilizing filter.

### Example 4—Process Flow for Preparation of ²²⁵Ac-Lintuzumab (²²⁵Ac-HuM195)

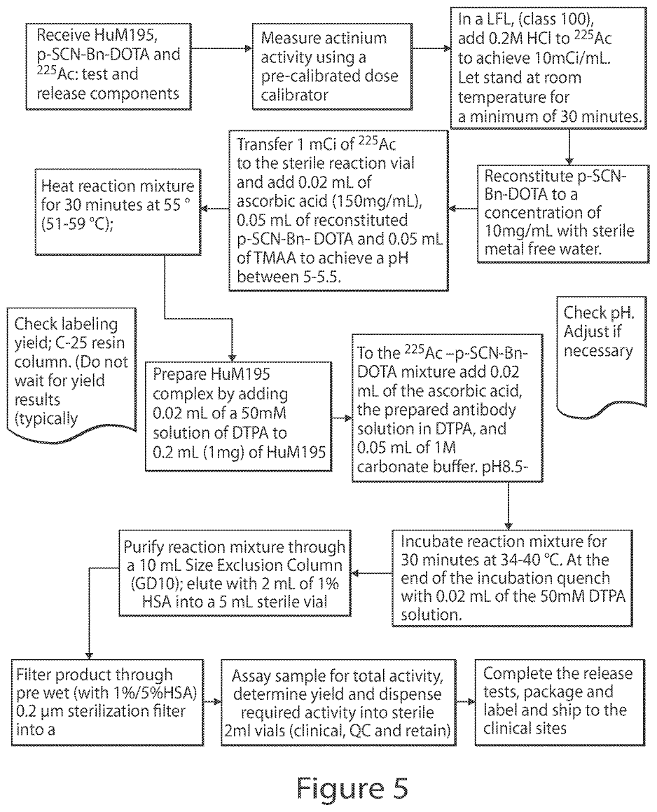

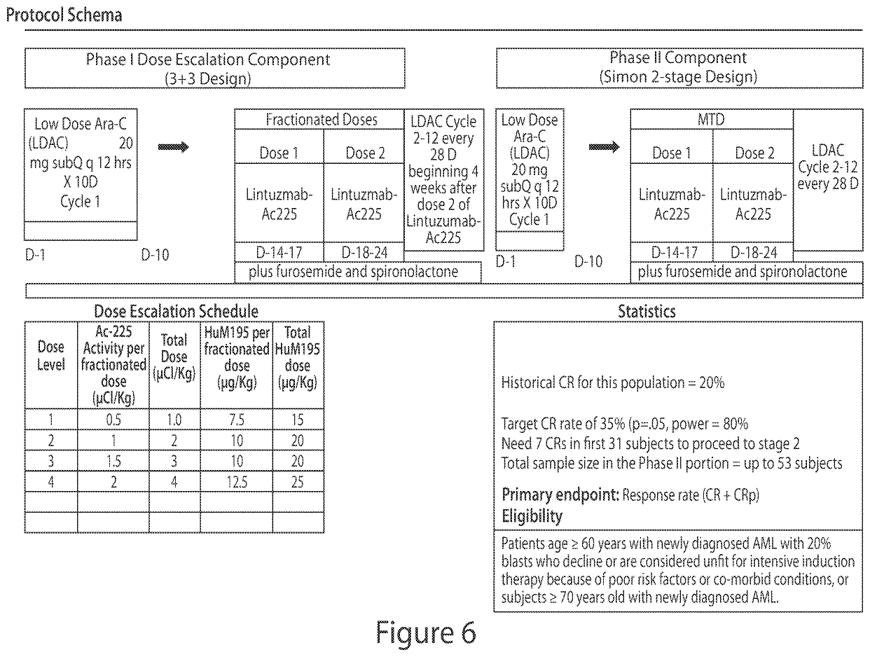

The procedure, shown in FIG. 5, begins with confirming the identity of all components and the subsequent QC release of the components to production. The ²²⁵Ac is assayed to confirm the level of activity and is reconstituted to the desired activity concentration with hydrochloric acid. A vial of lyophilized p-SCNBn-DOTA is reconstituted with metal-free water to a concentration of 10 mg/mL. To the actinium reaction vial, 0.02 ml of ascorbic acid solution (150 mg/mL) and 0.05 ml of reconstituted p-SCN-Bn-DOTA are added and the pH adjusted to between 5 and 5.5 with 2M tetramethylammonium acetate (TMAA). The mixture is then heated at 55±4° C. for 30 minutes.

To determine the labeling efficiency of the ²²⁵Ac-p-SCN-Bn-DOTA, an aliquot of the reaction mixture is removed and applied to a 1 ml column of Sephadex C25 cation exchange resin. The product is eluted in 2-4 ml fractions with a 0.9% saline solution. The fraction of ²²⁵Ac activity that elutes is ²²⁵Ac-p-SCN-Bn-DOTA and the fraction that is retained on the column is un-chelated, unreactive ²²⁵Ac. Typically, the labeling efficiency is greater than 95%.

To the reaction mixture, 0.22 ml of previously prepared HuM195 in DTPA (1 mg HuM195) and 0.02 ml of ascorbic acid are added. The DTPA is added to bind any trace amounts of metals that may compete with the labeling of the antibody. The ascorbic acid is added as a radio-protectant. The pH is adjusted with carbonate buffer to pH 8.5-9. The mixture is heated at 37±3° C. for 30 minutes. The final product is purified by size exclusion chromatography using 10DG resin and eluted with 2 ml of 1% HSA. Typical reaction yields are 10%.

### Example 5—Actimab-A Treatment in a Phase 1 Clinical Trial for Relapsed and Refractory AML Patients

Relapsed and refractory adult AML patients of all ages were enrolled in this single treatment group trial. Treatment consisted of a single infusion of Actimab-A administered at escalating doses as shown in Table 1 below.

TABLE 1

| | | |
|---|---|---|
| Dose Level | ²²⁵Ac Activity | HuM195 Dose |

| | | | |
|---|---|---|---|
| 1 | 0.5 | μCi/kg | 15 μg/kg |
| 2 | 1 | μCi/kg | 15 μg/kg |
| 3 | 2 | μCi/kg | 20 μg/kg |
| 4 | 4 | μCi/kg | 25 μg/kg |
| 5 | 3 | μCi/kg | 20 μg/kg |

Each cohort was planned for three patients. Dose escalation proceeded if none of the three patients experienced dose-limiting toxicities (DLTs). If one of the three patients experienced DLTs, the cohort was expanded with additional three patients. If none experienced DLTs, the dose escalation continued. If two out of six patients experienced DLTs, dose level was decreased. The total of 20 patients were treated and their baseline characteristics were collected, including circulating blasts counts. Circulating blasts counts were available for 18 patients. After the treatment, patients were followed up for safety and efficacy, including anti-leukemic effects, which primarily comprises blasts as a percentage of all cells in patients' red bone marrow.

### Example 6—Actimab-A Treatment in a Phase 1/2 Trial in Newly Diagnosed AML Patients

In the Phase 1 portion of the Actimab-A Phase 1/2 multicenter trial, newly diagnosed older AML patients ineligible for treatment with intensive chemotherapy regimens were treated with a course of low dose cytarabine followed by escalating fractionated doses (two fractions) of Actimab-A per Table 2 below. If the patient's disease did not progress after treatment with Actimab-A, and if the patient remained otherwise eligible, additional up to 11 low dose cytarabine cycles of treatment were to be administered.

TABLE 2

| | Ac-225 | | | |
|---|---|---|---|---|
| | Activity per | | HuM195 per | Total |
| | fractionated | Total | fractionated | HuM195 |
| Dose | dose | Dose | dose | dose |
| Level | (μCi/Kg) | (μCi/Kg) | (μg/Kg) | (μg/Kg) |
| 1 | 0.5 | 1.0 | 7.5 | 15 |
| 2 | 1 | 2 | 10 | 20 |
| 3 | 1.5 | 3 | 10 | 20 |
| 4 | 2 | 4 | 12.5 | 25 |

Each cohort was planned to enroll 3 patients. Dose escalation proceeded if none of the three patients experienced dose-limiting toxicities (“DLTs”). If one of the three patients experienced DLTs, the cohort was expanded with an additional three patients. If none experienced DLTs, the dose escalation continued. If two out of six patients experienced DLTs, the dose level was decreased. A total of 18 patients were treated and their baseline characteristics were collected, including circulating blast counts. Circulating blast counts were available for all 18 patients. After the treatment, patients were followed up for safety and efficacy, including anti-leukemic effects primarily comprising blasts as a percentage of all cells in patients' red bone marrow.

### Example 7—Efficacy Outcomes in an Actimab-A Phase 1 Trial for Relapsed and Refractory AML Patients and a Phase 1/2 Trial for Older Newly Diagnosed AML Patients

Initial anti-leukemic efficacy was analyzed as a function of achieving a composite complete response (“CRc”), where CRc is defined as achieving a post-treatment patient bone marrow blast percentage of ≤5%. Response rates per dose level in both referenced trials are shown in Tables 3 and 4 below. Table 3 shows efficacy outcomes for the Phase 1 single dose trial in relapsed and refractory AML patients.

TABLE 3
Patients in the Trial

| | Patients Treated | Achieved CRc | % CRc |
|---|---|---|---|
| Dose level 1 (0.5 μCi/kg) | 3 | 0 | 0% |
| Dose level 2 (1.0 μCi/kg) | 3 | 1 | 33% |
| Dose level 3 (2.0 μCi/kg) | 5 | 0 | 0% |
| Dose level 4 (3.0 μCi/kg) | 5 | 1 | 20% |
| Dose level 5 (4.0 μCi/kg) | 2 | 1 | 50% |

Table 4 shows efficacy outcomes for the Phase 1 fractionated dose trial in older newly diagnosed AML patients.

TABLE 4
Patients in the Trial

| | Patients | Achieved | |
|---|---|---|---|
| | Treated | CRc | % CRc |
| Dose level 1 (0.5 μCi/kg × 2) | 3 | 0 | 0% |
| Dose level 2 (1.0 μCi/kg × 2) | 6 | 1 | 17% |
| Dose level 3 (1.5 μCi/kg × 2) | 3 | 2 | 67% |
| Dose level 4 (2.0 μCi/kg × 2) | 6 | 2 | 33% |
| Total | 18 | 5 | 28% |

### Example 8—Efficacy Analysis by Commonly Used Risk Factors of an Actimab-A Phase 1/2 Trial for Newly Diagnosed AML Patients

There are several commonly used metrics used as prognostic factors to determine the likelihood of response in newly diagnosed AML. The most commonly used prognostic factors are age, cytogenetics and the presence of antecedent hematologic disease (most often myelodysplastic syndrome (MDS, in which case AML is referred to as secondary AML (“sAML”)). Those factors were applied to analyze efficacy outcomes in the Phase 1 older newly diagnosed AML trial. Results of those analyses are presented in Table 5 below.

TABLE 5

| | Responders | Non-responders |
|---|---|---|
| Age (Median) | 76 | 77 |
| Genetic risk | High | Intermediate |
| Response % | 29% | 27% |
| De novo vs sAML | De novo | sAML |
| Response % | 50% | 17% |

It was concluded that none of the above commonly used prognostic factors correlates with statistically significant differences in response outcomes.

### Example 9—Peripheral Blast Burden Correlation with Efficacy Outcomes in Actimab-A Phase 1 and Phase 1/2 Clinical Trials for AML Patients

Additional analyses were performed, and it was surprisingly discovered that the number of circulating blasts has a strong and statistically significant correlation with the clinical outcome. This discovery was observed in both trials, i.e., in both relapsed/refractory and newly diagnosed patients. A circulating blast count of fewer than 200 per microliter corresponded with responses. However, when the circulating blast count was greater than or equal to 200 per microliter, no patients responded. These data are presented in Table 6 below.

TABLE 6

| Circulating blasts per microliter | ≥200 | <200 |
|---|---|---|
| Phase 1 single dose trial | 0% | 43% |
| Phase ½ fractionated dosing trial | 0% | 42% |

### Example 10—Responses in Newly Diagnosed and Relapsed/Refractory AML Patients According to Peripheral Blast Status Prior to Actimab-A Treatment

Response outcomes in AML patients were compared by dose level in the two referenced trials by selecting only patients with <200 circulating blasts per microliter of peripheral blood. Tables 7-9 below summarize these findings.

Table 7 shows efficacy outcomes per peripheral blast counts by dose level in the Phase 1 trial for relapsed and refractory AML patients.

TABLE 7

| | Pts | Pts with | | Pts | Pts with | |
|---|---|---|---|---|---|---|
| | pb ≥ 200 | CRc | % CRc | pb < 200 | CRc | % CRc |
| Dose level 1 (0.5 μCi/kg) | 2 | 0 | 0% | 1 | 0 | 0% |
| Dose level 2 (1 μCi/kg) | 0 | 0 | 0% | 3 | 1 | 33% |
| Dose level 3 (2 μCi/kg) | 5 | 0 | 0% | 0 | 0 | NA |
| Dose level 4 (3 μCi/kg) | 3 | 0 | 0% | 2 | 1 | 50% |
| Dose level 5 (4 μCi/kg) | 1 | 0 | 0% | 1 | 1 | 100% |
| Total | 11 | 0 | 0% | 7 | 3 | 43% |

Table 8 shows efficacy outcomes per peripheral blast counts by dose level in the Phase 1/2 trial for newly diagnosed older AML patients.

TABLE 8

| | Pts | Pts with | | Pts | Pts with | |
|---|---|---|---|---|---|---|
| | pb ≥ 200 | CRc | % CRc | pb < 200 | CRc | % CRc |
| Dose level 1 (0.5 μCi/kg × 2) | 1 | 0 | 0% | 2 | 0 | 0% |
| Dose level 2 (1 μCi/kg × 2) | 3 | 0 | 0% | 3 | 1 | 33% |
| Dose level 3 (1.5 μCi/kg × 2) | 0 | 0 | 0% | 3 | 2 | 67% |
| Dose level 4 (2 μCi/kg × 2) | 2 | 0 | 0% | 4 | 2 | 50% |
| Total | 6 | 0 | 0% | 12 | 5 | 42% |

Table 9 shows a statistical analysis of the responses in both AML trials.

TABLE 9

| Patients with: | CRc rates | CRc % |
|---|---|---|
| <200 pb | 8/19 | 42% |
| ≥200 pb | 0/17 | 0% |

* p value: 0.002416 (highly significant)

### Example 11—Anti-Leukemic Effect of Actimab-A Based on Significant Leukemic Blast Cell Killing

In early stage clinical trials, anti-leukemic effect is often measure as the ability of the studied therapy to reduce leukemic blast burden in the bone marrow by ≥50%. The ability of Actimab-A to have this effect was analyzed by comparing patients with peripheral blast burdens of ≥200 and patients with peripheral blast burdens of <200 per microliter of peripheral blood. Results of these analyses are presented in Table 10 below. These results represent analyses of 38 patients wherein data were available for 31 of them.

TABLE 10

| | ≥50% reduced | <50% reduced |
|---|---|---|
| | patients (%) | patients (%) |
| pb < 200 | 12 (80%) | 3 (20%) |
| pb ≥ 200 | 4 (25%) | 12 (75%) |

* p value: 0.002197 (highly significant)

### REFERENCES

1. Burnett, et al., “A Comparison of Low-Dose Cytarabine and Hydroxyurea With or Without All-trans Retinoic Acid for Acute Myeloid Leukemia and High-Risk Myelodysplastic Syndrome in Patients Not Considered Fit for Intensive Treatment”, Cancer, Mar. 15, 2007, Vol. 109, No 6.
2. Co, et al., J. Immunol. 148:1149-1154, 1992.
3. Domino and Baldor, “The 5-Minute Clinical Consult 2014.”
4. Gansow, et al., U.S. Pat. No. 4,923,985.
5. Harousseau, et al., “A randomized phase 3 study of tipifarnib compared with best supportive care, including hydroxyurea, in the treatment of newly diagnosed acute myeloid leukemia in patients 70 years or older”, Blood, 6 Aug. 2009, Vol. 114, No. 6.
6. Jurcic, et al., “Phase I Trial of Targeted Alpha-Particle Therapy with Actinium-225 (²²⁵Ac)-Lintuzumab and Low-Dose Cytarabine (LDAC) in Patients Age 60 or Older with Untreated Acute Myeloid Leukemia (AML)”, ASH 2016 Abstract.
7. Kumar, “Genetic Abnormalities and Challenges in the Treatment of Acute Myeloid Leukemia”, Genes & Cancer 2 (2): 95-107, 2011.
8. McDevitt, “Design and synthesis of ²²⁵Ac radioimmunopharmaceuticals”, Applied Radiation and Isotope, 57 (2002), 841-847.
9. Mulford et al., “The Promise of Targeted α-Particle Therapy”, The Journal of Nuclear Medicine, Vol. 46, No. 1 (Suppl), January 2005.
10. Mylotarg Wyeth Product Monograph (2005).
11. Pollard, et al., “Correlation of CD33 expression level with disease characteristics and response to gemtuzumab ozogamicin containing chemotherapy in childhood AML”, Blood, 2012 April 19, 119 (16): 3705-3711.
12. V. H. J. van der Velden, et al., “High CD33-antigen loads in peripheral blood limit the efficacy of gemtuzumab ozogamicin (Mylotarg) treatment in acute myeloid leukemia patients”, Leukemia, 2004 May, 18 (5): 983-8.
13. Scheinberg, et al., U.S. Pat. No. 6,683,162.
14. SWOG Oncology Research Professional (ORP) Manual, Volume I, Chapter 11A, Leukemia (2014).

1.-29. (canceled)

30. A method for treating relapsed and/or refractory acute myeloid leukemia in a human subject, comprising the step of:
intravenously administering a therapeutically effective dose of lintuzumab labeled with an alpha particle-emitting isotope to a human subject afflicted with relapsed and/or refractory acute myeloid leukemia, wherein the human subject has a peripheral blast burden below 200 blast cells/μl.

31. The method of claim 30, further comprising the step of before administering the labeled lintuzumab to the human subject, determining that the human subject has a peripheral blast burden below 200 blast cells/μl.

32. The method of claim 30, wherein the human subject is ineligible to receive high intensity chemotherapy.

33. The method of claim 31, wherein the human subject is ineligible to receive high intensity chemotherapy.

34. The method of claim 30, further comprising the step of:
before administering the labeled lintuzumab to the human subject, administering low dose cytarabine to the human subject.

35. The method of claim 31, further comprising the step of:
before administering the labeled lintuzumab to the human subject, administering low dose cytarabine to the human subject.

36. The method of claim 32, further comprising the step of:
before administering the labeled lintuzumab to the human subject, administering low dose cytarabine to the human subject.

37. The method of claim 33, further comprising the step of:
before administering the labeled lintuzumab to the human subject, administering low dose cytarabine to the human subject.

38. The method of claim 30, wherein the human subject's peripheral blast burden is 50 blast cells/μl or lower.

39. The method of claim 30, further comprising the step of:
before administering the labeled lintuzumab to the human subject, medically lowering the human subject's peripheral blast burden from at or above 200 blast cells/μl to below 200 blast cells/μl,
wherein the administering step is performed while the human subject's peripheral blast burden is still below 200 blast cells/μl.

40. The method of claim 39, wherein the human subject's peripheral blast burden is medically lowered to 50 blast cells/μl or lower.

41. The method of claim 34, further comprising the step of:
before administering the labeled lintuzumab to the human subject, medically lowering the human subject's peripheral blast burden from at or above 200 blast cells/μl to below 200 blast cells/μl,
wherein the administering step is performed while the human subject's peripheral blast burden is still below 200 blast cells/μl.

42. The method of claim 41, wherein the human subject's peripheral blast burden is medically lowered to 50 blast cells/μl or lower.

43. A method for treating acute myeloid leukemia in a human subject, comprising the step of:
intravenously administering a therapeutically effective dose of lintuzumab labeled with an alpha particle-emitting isotope to a human subject afflicted with acute myeloid leukemia, wherein the human subject has a peripheral blast burden below 200 blast cells/μl.

44. The method of claim 43, further comprising the step of before administering the labeled lintuzumab to the human subject, determining that the human subject has a peripheral blast burden below 200 blast cells/μl.

45. The method of claim 43, wherein the human subject is ineligible to receive high intensity chemotherapy.

46. The method of claim 44, wherein the human subject is ineligible to receive high intensity chemotherapy.

47. The method of claim 43, further comprising the step of:
before administering the labeled lintuzumab to the human subject, administering low dose cytarabine to the human subject.

48. The method of claim 44, further comprising the step of:
before administering the labeled lintuzumab to the human subject, administering low dose cytarabine to the human subject.

49. The method of claim 45, further comprising the step of:
before administering the labeled lintuzumab to the human subject, administering low dose cytarabine to the human subject.


SEQUENCE LISTING (4 sequences)

SEQ ID NO: 1 - DNA, 1952 bp, synthetic construct
  misc_feature 1..1952: note = Description of Artificial Sequence: Synthetic polynucleotide
  source 1..1952: mol_type = other DNA
  CDS 12..407
  CDS 766..1083
tctagaccaccatggagaaagacacactcctgctatgggtcctacttctctgggttccaggttccacaggtgacattcagatgacccagtctccgagctctctgtccgcatcagtaggagacagggtcaccatcacatgcagagccagcgaaagtgtcgacaattatggcattagctttatgaactggttccaacagaaacccgggaaggctcctaagcttctgatttacgctgcatccaaccaaggctccggggtaccctctcgcttctcaggcagtggatctgggacagacttcactctcaccatttcatctctgcagcctgatgacttcgcaacctattactgtcagcaaagtaaggaggttccgtggacgttcggtcaagggaccaaggtggagatcaaacgtaagtagaatccaaagtctagaaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgctctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgttttaccaactactcaatttctcttataagggactaaatatgtagtcatcctaaggcgcataaccatttataaaaatcatccttcattctattttaccctatcatcctctgcaagacagtcctccctcaaacccacaagccttctgtcctcacagtcccctgggccatggtaggagagacttgcttccttgttttcccctcctcagcaagccctcatagtcctttttaagggtgacaggtcttacagtcatatatcctttgattcaattccctgagaatcaaccaaagcaaatttttcaaaagaagaaacctgctataaagagaatcattcattgcaacatgatataaaataacaacacaataaaagcaattaaataaacaaacaatagggaaatgtttaagttcatcatggtacttagacttaatggaatgtcatgccttatttacatttttaaacaggtactgagggactcctgtctgccaagggccgtattgagtactttccacaacctaatttaatccacactatactgtgagattaaaaacattcattaaaatgttgcaaaggttctataaagctgagagacaaatatattctataactcagcaatcccacttctaggatcc

SEQ ID NO: 2 - protein, 238 aa, synthetic construct
  REGION 1..238: note = Description of Artificial Sequence: Synthetic polypeptide
MEKDTLLLWVLLLWVPGSTGDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 3246 bp, synthetic construct
  misc_feature 1..3246: note = Description of Artificial Sequence: Synthetic polynucleotide
  source 1..3246: mol_type = other DNA
  CDS 12..416
  CDS 660..953
  CDS 1345..1389
  CDS 1508..1837
  CDS 1935..2255
tctagaccaccatgggatggagctggatctttctcttcctcctgtcaggaactgctggcgtccactctcaggttcagctggtgcagtctggagctgaggtgaagaagcctgggagctcagtgaaggtttcctgcaaagcttctggctacaccttcactgactacaacatgcactgggtgaggcaggctcctggccaaggcctggaatggattggatatatttatccttacaatggtggtaccggctacaaccagaagttcaagagcaaggccacaattacagcagacgagagtactaacacagcctacatggaactctccagcctgaggtctgaggacactgcagtctattactgcgcaagagggcgccccgctatggactactggggccaagggactctggtcactgtctcttcaggtaagaatggcctctagaccaccatgggatggagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagcccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagggaggcagagcgggtcccactgtccccacactggcccaggctgtgcaggtgtgcctgggccgcctagggtggggctcagccaggggctgccctcggcagggtgggggatttgccagcgtggccctccctccagcagcacctgccctgggctgggccacgggaagccctaggagcccctggggacagacacacagcccctgcctctgtaggagactgtcctgttctgtgagcgccctgtcctccgacctccatgcccactcgggggcatgcctagtccatgtgcgtagggacaggccctccctcacccatctacccccacggcactaacccctggctgccctgcccagcctcgcacccgcatggggacacaaccgactccggggacatgcactctcgggccctgtggagggactggtgcagatgcccacacacacactcagcccagacccgttcaacaaaccccgcactgaggttggccggccacacggccaccacacacacacgtgcacgcctcacacacggagcctcacccgggcgaactgcacagcacccagaccagagcaaggtcctcgcacacgtgaacactcctcggacacaggcccccacgagccccacgcggcacctcaaggcccacgagcctctcggcagcttctccacatgctgacctgctcagacaaacccagccctcctctcacaagggtgcccctgcagccgccacacacacacaggggatcacacaccacgtcacgtccctggccctggcccacttcccagtgccgcccttccctgcaggatcc

SEQ ID NO: 4 - protein, 465 aa, synthetic construct
  REGION 1..465: note = Description of Artificial Sequence: Synthetic polypeptide
MGWSWIFLFLLSGTAGVHSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK